(19) United States
(12) Patent Application Publication
(10) Pub. No.: US 2021/0395306 A1
(43) Pub. Date: Dec. 23, 2021

(54) POLYPEPTIDES FOR THE TREATMENT OF STRESS, IMMUNOREACTION AND STROKE SYNDROMES

(71) Applicant: Neopep Pharma GmbH & Co. KG, Hannover (DE)

(72) Inventor: Wolf-Georg FORSSMANN, Hannover (DE)

(21) Appl. No.: 17/292,297

(22) PCT Filed: Nov. 8, 2019
(86) PCT No.: PCT/EP2019/080685

§ 371 Date: May 7, 2021

(30) Foreign Application Priority Data
Nov. 9, 2018 (EP) 18205455.1

(51) Int. Cl. C07K 7/08; A61P 35/00

(52) U.S. Cl. CPC C07K 7/08; A61K 38/00; A61P 35/00

(57) ABSTRACT

A polypeptide having the general amino acid sequence written in the single letter code

Z¹X¹X²X³X⁴X⁵QX⁷APX¹⁰X¹¹SZ³,
wherein
   X¹=V, M or L, in particular L,
   X²=V, L or M, in particular V,
   X³=R, K or C, in particular R,
   X⁴=Y or W, in particular Y,
   X⁵=S or T, in particular T,
   X⁷=K, C or R, in particular K,
   X¹⁰=Q or C, in particular Q, and
   X¹¹=V, M or F, in particular V
   and
   Z³=0, or Z⁴, wherein
   Z⁴=0 or is a modification of the C-terminal carboxyl group of the peptide chain, which modification forms together with the carboxyl group of the C-terminal amino acid of the peptide a moiety having the structure —C(O)—O—R¹ or —C(O)—NR²R³, wherein R¹ is a substituted or unsubstituted alkyl, aryl, aralkyl, cyclo alkyl and heterocyclo alkyl group.

The present invention concerns polypeptides and their therapeutic uses especially for the treatment of stress, immunoreaction and stroke syndromes.

### BACKGROUND OF THE INVENTION

When cells migrate in the body, they are guided by chemokines. Cells that are attracted by chemokines follow a signal of increasing chemokine concentration towards the source of the chemokine. There are currently four different chemokine families known which differ on the spacing of their first two cysteine residues. The so called CXC chemokines or α-chemokines comprise one amino acid a s spacer between the two cysteines, represented in the name with “X”.

Accordingly, due to chemokine receptors on the surface of the cells the chemokines influence the cells. The chemokine receptors are integral membrane proteins. The CXC chemokine receptor, and especially the CXC chemokine receptor 4 (CXCR4) activates rapid growth in cancer cells and migration, forming metastases throughout the body, preferentially in the lung, bone and liver. Also, HIV can use CXCR4 to infect CD4+ T cells.

CXCR4 is involved in multiple developmental and physiological processes including stem cell homing (Mohle and Drost, 2012) and migration of immune cells (Campbell et al., 2003). Man-made CXCR4 antagonists are capable of mobilizing hematopoietic stems cells (HSCs), which are utilized for immune reconstitution after organ transplantation or chemotherapy (Ratajczak and Kim, 2012; Schroeder and DiPersio, 2012). In addition, CXCR4 is also a major co-receptor for HIV-1 entry into target cells (Feng et al., 1996; Bleul et al., 1996). Co-receptor utilization of CXCR4 is highly effective and a high proportion of CD4+ T cells express this GPCR in lymphatic tissues in vivo. Nonetheless, almost exclusively HIV-1 variants utilizing the C—C chemokine receptor type 5 (CCR5) are transmitted and found during chronic HIV-1 infection (Alkhatib et al., 1996; Deng et al., 1996; Dragic et al., 1996). It has been proposed that multiple factors contribute to the inefficient transmission of CXCR4-tropic (X4) HIV-1 strains (Margolis and Shattock, 2006). However, the mechanism(s) underlying the effective control of X4 HIV-1 in immunocompetent individuals remain poorly understood.

Research on CXCR4-antagonists has recently become an immense field of projects due to the manifold indications. In particular the efforts to find a strategy to intervene with cancer cell proliferation, differentiation, and metastasis was not so successful in clinical studies yet as expected. The development of one of the compound groups, namely AMD3100, a CXCR4-antagonists (a bicyclame compound: Hendrix and Flexner 2000), had to be stopped for long term treatments due to toxic side effects. Although AMD3100 is registered for single short applications in stem cell mobilization, it is nevertheless a challenge to find adequate antagonists to the target CXCR4.

Zirafi, O. et al. report in J. Leukoc. Biol. 99: 663-868; 2016 about an endogenous peptide inhibitor of CXCR4 as an antagonist of CXCR4. This peptide is a 16-residue fragment of human serum albumin and was isolated as an inhibitor of CXCR4-tropic human immunodeficiency virus type 1 from a blood-derived peptide library. Endogenous peptide inhibitor of CXCR4 binds the second extracellular loop of CXCR4, thereby preventing engagement of CXCL12 and antagonizing the receptor. Consequently, endogenous peptide inhibitor of CXCR4 inhibits CXCL12-mediated migration of CXCR4-expressing cells in vitro, mobilizes hematopoietic stem cells, and suppresses inflammatory responses in vivo. The authors discuss the generation of endogenous peptide inhibitor of CXCR4, its relevance as biomarker for disease, and its role in human immunodeficiency virus/acquired immunodeficiency syndrome pathogenesis and cancer.

WO 2009/004054 A2 discloses a peptide having the amino acid sequence Z1-LVRYTKKVPQVSTPTL-Z2 (ALB-408) and its biologically active fragments and/or variants and/or derivatives, especially amidated, acetylated, sulfated, phosphorylated and/or glycosylated derivatives, and peptides obtainable by multiple synthesis which have the biological activity of ALB408-423; wherein Z represents number of from 0 to 10 amino acid residues.

WO 2014/198834 A1 discloses peptides, in particular dimers, effective in blocking the CXC-chemokine receptor 4 (CXCR4) mediated HIV-1 NL4-3 (X4-tropic) infection with an IC50 value of less than 50 μM.

It is therefore an object of the invention to provide peptides with antagonistic activities against natural CXCR4. Especially, the peptides should inhibit proliferation of cancer cells, metastasis and show the types of cancers which are addressed by the different analogs and reaction of antiinflammatory allergic reactions.

Another object of the invention is to provide a compound which is capable to reduce the receptor activity of CXCR4.

Another object of the present invention is to provide a compound which is capable to influence proliferation of a cancer cell.

Another object of the present invention is to provide a compound which is capable to influence migration or homing of a cancer cell.

A still further object of the present invention is to provide a compound which is capable to influence the formation of metastases.

Another object of the present invention is to provide a compound which is capable to treat highly aggressive tumors so that the cancer is considerably inhibited or becomes a chronic disease.

Still another object of the present invention is to provide a compound which is capable to regulate and treat various diseases, such as immune and allergic diseases, tissue growth and nervous system regulation.

### SUMMARY OF THE INVENTION

The objects of the invention are solved by any of the polypeptides of the invention. The polypeptides of the invention exhibit great therapeutic potential.

A polypeptide of the invention comprises the general amino acid sequence (written in the single letter code)

Z¹ X¹ X² X³ X⁴ X⁵ X⁶ X⁷ A P X¹⁰ S Z³,

wherein
X¹=V, M or L, in particular L,
X²=V, L or M, in particular V,
X³=R, K or C, in particular R,
X⁴=Y or W, in particular Y,
X⁵=S or T, in particular T,
X⁶=Q or C, in particular Q,
X⁷=K, C or R, in particular K,
X¹⁰=Q or C, in particular Q, and
X¹¹=V, M or F, in particular V.
   and
   Z¹=0, Z², or pyro glutamate, wherein
   Z²=0 or is a modification of the N-terminal nitrogen atom of the peptide chain which modification forms together with the amino group of the N-terminal amino acid of the peptide a moiety having the structure —NR²R³ wherein R² and/or R³ are independently from each other H or a substituted or unsubstituted acyl alkyl, aryl, aralkyl, cyclo alkyl and heterocyclo alkyl group;
   Z³=0, or Z⁴, wherein
   Z⁴=0 or is a modification of the C-terminal carboxyl group of the peptide chain, which modification forms together with the carboxyl group of the C-terminal amino acid of the peptide a moiety having the structure —C(O)—O—R¹ or —C(O)—NR²R³, wherein R¹ is a substituted or unsubstituted alkyl, aryl, aralkyl, cyclo alkyl and heterocyclo alkyl group.

When it is stated, that the group(s) Z¹ and/or Z² and/or Z³ and/or Z⁴=0, it means within the present invention that the respective group is not present in this specific embodiment.

Surprisingly it was found that a polypeptide with a nonpolar protein at one chain end (X¹) and a polar protein at the other chain end (S) shows antagonistic activities against natural CXCR4.

In a preferred embodiment, X⁶ is Q so that the invention refers to a polypeptide comprising the general amino acid sequence (written in the single letter code)

Z¹ X¹ X² X³ X⁴ X⁵ Q X⁷ A P X¹⁰ X¹¹ S Z³,

in the polypeptide of the present invention the peptides are selected from the following:
X¹=V, M or L, in particular L,
X²=V, L or M, in particular V,
X³=R, K or C, in particular R,
X⁴=Y or W, in particular Y,
X⁵=S or T, in particular T,
X⁶=K, C or R, in particular K,
X¹⁰=Q or C, in particular Q, and
X¹¹=V, M or F, in particular V.

In a preferred embodiment of the invention the peptide of the invention comprises at least one of the following amino acid sequences:

LVRYTQKAPQVS, MVRYTQKAPQVS, VVRYTQKAPQVS,
LMRYTQKQPQVS, LLRYTQKQPQVS, LVKYTQKQPQVS,
LVCYTQKQPQVS, LVRWTQKAPQVS, LVRYSQKQPQVS,
LVRYTCKAPQVS, LVRYTQCAPQVS, LVRYTQRAPQVS,
LVRYTQKAPCVS, LVRYTQKAPQMS, or LVRYTQKAPQFS.

The sequence LVRYTQKAPQVS is a particularly preferred embodiment.

According to the invention single or several amino acid residues in the sequence can been exchanged, deleted or added, or chemical modifications on single amino acids of said polypeptide can been introduced which result in an improved biological or pharmacological activity of the unmodified polypeptide of the invention. Respective methods for modifications are known to the skilled person.

Furthermore at least one side chain of an amino acid of said polypeptide can be chemically modified, in particular phosphorylated, amidated, acetylated, glycosylated, PEGylated, HESylated or combinations thereof.

The choice of the suitable functional group for the PEG derivative is based on the type of available reactive groups on the molecule that will be coupled to the PEG. For proteins, typical reactive amino acids include lysine, cysteine, histidine, arginine, aspartic acid, glutamic acid, serine, threonine, tyrosine. The N-terminal amino group and the C-terminal carboxylic acid can also be used as a specific site by conjugation with aldehyde functional polymers.

According to the invention the polypeptide according of the invention may comprise at least one D-amino acid. In particular, the polypeptide of the invention may be composed by a chain of D-amino acids in a retro-inverso configuration of the chain of the polypeptide of the invention.

Thus, also retro-inverso peptides of the peptides of the invention are in the scope of the present invention, as well as other derivatives stabilizing the peptide bond against peptidases. The term derivative means within the scope of the present application all length fragments including truncations at the N and C terminus, the peptide of the invention containing amino acid residue substitutions including D-amino acid residues and modified amino acid residues as well as peptides containing disulfide bonds and extension at the N and C terminus.

A further subject matter of the present invention is a medicament comprising at least one polypeptide of the invention and a pharmaceutically acceptable carrier. In one of the simplest embodiments the polypeptide of the invention can be administered in water for infusion, physiological saline, or buffered aqueous solutions. Also, other formulations are possible for example encapsulation in liposomes forming nanoparticles of various sizes, for example from 20 to 2.000 nm.

Typically, the peptide of the invention is administered in amounts of 10-1,000 mg/kg body weight for a time period sufficient to stop tumor growth. The time of administration is usually between two to ten weeks. The medicament of the invention is preferably suitable for oral, intravenous, intramuscular, intracutaneous, subcutaneous, intrathecal administration or present in form of an aerosol suitable for transpulmonary and intranasal administration, in particular encapsulated in liposomes; or for use in aqueous or liposomal packaging.

Subject matter of the present invention is also a polynucleotide coding for a polypeptide of the invention and/or its fragments, variants, derivatives and analogues. The polynucleotide of the invention may be constituted of DNA, RNA, genomic DNA or PNA. A polynucleotide hybridizing to a polynucleotide according to the invention that codes for a polypeptide of the invention is also subject of the present invention.

A further subject matter of the present invention is a vector containing a polynucleotide according to the invention, as well as a genetically engineered host cell containing the vector according the invention.

A still further subject matter of the present invention is an antibody directed against at least one polypeptide of the invention.

Yet another subject matter of the present invention is an antagonist/inhibitor compound directed against a polypeptide according to the invention.

Subject matter of the invention is also a peptide of the invention for use in the treatment of neurological diseases, in particular stroke, Parkinson's disease, Alzheimer's disease, multiple sclerosis; in the field of immunology in particular for the treatment of the WHIM-syndrome, lupus erythematosus and rheumatoid arthritis; in the field of oncology in particular for the treatment of cancers, in particular cancers showing the CRCX receptor such as cancer of the liver, pancreas, prostate, or breast cancer; for the treatment of lack of mobilization, proliferation and migration of stem cells, T-cell activation as well as support of immunoblasts such as CTL/PD-1; in the treatment of wounds caused by burning; for the treatment of antifibrosis; treatment or prevention of scars; for treatment of cardiologic disorders, in particular heart insufficiency; for the treatment of metabolic disorders, in particular diabetes; for the treatment of viral diseases, in particular infections with HIV-I, HIV-2, Cytomegalo virus, Herpes simplex virus (type 1 and 2), Varicella zoster virus, Hepatitis A and Hepatitis B virus, Influenza virus, Polio virus, Rhino virus, Rubella virus, Measles virus, Rabies virus, Rous sarcoma virus, Epstein-Barr Virus; and for the treatment of infections caused by bacteria and fungi, in particular Pseudomonas, Candida, S. aureus; for the treatment of infectious processes, abnormal infectious processes; treatment of growth disorders, treatment of neuronal diseases, disorders of the blood clotting cascade and hematopoiesis, vascular diseases, diseases of the immune system, and for improving wound and bone healing, pulmonary disorders, and allergies.

Another process for the manufacturing of a polypeptide according to the invention is solid-phase synthesis in terms of Merrifield synthesis or liquid-phase synthesis by methods known to the skilled person using protected amino acids, and its purification.

Still another process for the manufacturing of a polypeptide according to the invention can employ methods of heterologous expression known to the skilled person using common biotechnological vectors, and if necessary subsequent posttranslational or chemical modification.

Subject matter of the present invention is a diagnostic agent containing poly- or monoclonal antibodies according to the invention or containing the nucleic acid or mRNA coding for a polypeptide according to the invention.

A further subject matter of the invention is a diagnostic agent containing a polypeptide according to the invention or a polynucleotide according to the invention for test systems for assaying tissue, plasma, urine and cerebrospinal fluid levels of this substance, diagnostic agents and test systems detecting a polypeptide according to the invention for assaying tissue, plasma, urine and cerebrospinal fluid levels of this substance by means of mass-spectrometric methods, such as MALDI-MS or ESI-MS, in connection with sample preparation by RP-HPLC, protein precipitation and/or solid-phase extraction. Preferably methods of mass spectrometry are used for the detection of minute quantities of the molecules in the range of femto or atto molar quantities.

Also, a diagnostic agent is subject of the invention which are containing a polypeptide according to the invention as markers for viral diseases, bacterial and fungal infections, inflammatory and neoplastic processes, and as markers in inflammatory processes, disturbed inflammation reactions, tumor diseases, growth disorders, diseases of the immune system, WHIm-syndrom, lupus erythematosus and as markers in bone diseases as well as others.

The invention is further described in more detail using the peptide LVRYTQKAPQVS (SEQ ID No. 1) as a typical representative of the peptide of the invention.

### EXAMPLES

### Peptides

The peptide of SEQ ID No. 1 and various derivates thereof were synthesized by conventional solid-phase synthesis on a peptide synthesizer 9050 (Applied Biosystems) using Fmoc chemistry. The peptide was purified by RP chromatography, and its identity and purity were established by analytical RP-HPLC and MALDI-MS and LC-ESI-MS.

### Cancer Cell Invasion Assay

The cancer cell invasion assay was performed on humanized rats (Eyol, E. et al., Oncology Reports, 28: 2177-2187, 2012). Pancreas carcinoma cells were implanted. The successful implant was observed by luminescent imaging as well as by an increase in CXCR4 expression.

After a successful implant of carcinoma cells, the rats were treated with a peptide according to SEQ ID No. 1. The results after 1 week of therapy (1 w of therapy), after 2 weeks of therapy (2 w of therapy) and 2 weeks after end of therapy are depicted in the below table.

### Result for Peptide of SEQ ID No. 1

| Amount of | | | 2 w after |
|---|---|---|---|
| peptide | 1 w of therapy | 2 w of therapy | end of therapy |
| 0 mg (control) | Tumor growth | Tumor growth | Tumor growth |
| 10 mg | No tumor growth | Tumor growth | Tumor growth |
| 20 mg | No tumor growth | Complete | Complete |
| | | Remission | Remission |
| 70 mg | No tumor growth | Remission | Complete |
| | | | Remission |

Rats without therapy died after tumor implant within few days.

Rats with therapy using the peptide of the present invention survived over at least two weeks. The tumor growth was stopped and depending on the concentration a partial or even complete remission of the tumor was observed. No toxic effect of the peptides was observed.

SEQUENCE LISTING
SEQ ID No. 1
Leu Val Arg Tyr Thr Gln Lys Ala Pro Gln Val Ser
SEQ ID No. 2
Met Val Arg Tyr Thr Gln Lys Ala Pro Gln Val Ser
SEQ ID No. 3
Val Val Arg Tyr Thr Gln Lys Ala Pro Gln Val Ser
SEQ ID No. 4
Leu Met Arg Tyr Thr Gln Lys Gln Pro Gln Val Ser
SEQ ID No. 5
Leu Leu Arg Tyr Thr Gln Lys Gln Pro Gln Val Ser
SEQ ID No. 6
Leu Val Lys Tyr Thr Gln Lys Gln Pro Gln Val Ser
SEQ ID No. 7
Leu Val Cys Tyr Thr Gln Lys Gln Pro Gln Val Ser
SEQ ID No. 8
Leu Val Arg Trp Thr Gln Lys Ala Pro Gln Val Ser
SEQ ID No. 9
Leu Val Arg Tyr Ser Gln Lys Gln Pro Gln Val Ser
SEQ ID No. 10
Leu Val Arg Tyr Thr Cys Lys Ala Pro Gln Val Ser
SEQ ID No. 11
Leu Val Arg Tyr Thr Gln Cys Ala Pro Gln Val Ser
SEQ ID No. 12
Leu Val Arg Tyr Thr Gln Arg Ala Pro Gln Val Ser
SEQ ID No. 13
Leu Val Arg Tyr Thr Gln Lys Ala Pro Cys Val Ser
SEQ ID No. 14
Leu Val Arg Tyr Thr Gln Lys Ala Pro Gln Met Ser
SEQ ID No. 15
Leu Val Arg Tyr Thr Gln Lys Ala Pro Gln Phe Ser

1. A polypeptide or a polynucleotide coding for the polypeptide having the general amino acid sequence written in the single letter code
Z¹ X¹ X² X³ X⁴ X⁵ Q X⁷ A P X¹⁰ X¹¹ S Z³,
wherein
X¹=V, M or L,
X²=V, L or M,
X³=R, K or C,
X⁴=Y or W,
X⁵=S or T,
X⁷=K, C or R,
X¹⁰=Q or C, and
X¹¹=V, M or F,
and
Z¹=0, Z², or pyro glutamate, wherein
Z²=0 or is a modification of the N-terminal nitrogen atom of the peptide chain which modification forms together with the amino group of the N-terminal amino acid of the peptide a moiety having the structure —NR²R³ wherein R² and/or R³ are independently from each other H or a substituted or unsubstituted acyl alkyl, aryl, aralkyl, cyclo alkyl and heterocyclo alkyl group;
Z³=0, or Z⁴, wherein
Z⁴=0 or is a modification of the C-terminal carboxyl group of the peptide chain, which modification forms together with the carboxyl group of the C-terminal amino acid of the peptide a moiety having the structure —C(O)—O—R¹ or —C(O)—NR²R³, wherein R¹ is a substituted or unsubstituted alkyl, aryl, aralkyl, cyclo alkyl and heterocyclo alkyl group.

2. The polypeptide or polynucleotide coding for the polypeptide according to claim 1, selected from the group consisting of polypeptides having or consisting of the amino acid sequence
LVRYTQKAPQVS, MVRYTQKAPQVS, VVRYTQKAPQVS,
LMRYTQKQPQVS, LLRYTQKQPQVS, LVKYTQKQPQVS,
LVCYTQKQPQVS, LVRWTQKAPQVS, LVRYSQKQPQVS,
LVRYTCKAPQVS, LVRYTQCAPQVS, LVRYTQRAPQVS,
LVRYTQKAPCVS, LVRYTQKAPQMS, or LVRYTQKAPQFS.

3. The polypeptide or polynucleotide coding for the polypeptide according to claim 1, wherein single or several amino acid residues in the sequence have been exchanged, deleted or added, or chemical modifications on single amino acids of said polypeptide have been introduced which result in an improved biological or pharmacological activity of the unmodified polypeptide.

4. The polypeptide or polynucleotide coding for the polypeptide according to claim 1, wherein at least one side chain of an amino acid of said polypeptide is phosphorylated, amidated, acetylated, glycosylated, PEGylated, HESylated or combinations thereof.

5. The polypeptide or polynucleotide coding for the polypeptide according to claim 1, wherein the polypeptide comprises at least one D-amino acid.

6. A composition selected from the group consisting of a medicament a vector, a genetically engineered host cell containing the vector, or a diagnostic agent, comprising at least one polypeptide or polynucleotide coding for the polypeptide;
the at least one polypeptide having the general amino acid sequence written in the single letter code
Z¹ X¹ X² X³ X⁴ X⁵ Q X⁷ A P X¹⁰ X¹¹ S Z³,
wherein
X¹=V, M or L,
X²=V, L or M,
X³=R, K or C,
X⁴=Y or W,
X⁵=S or T,
X⁷=K, C or R,
X¹⁰=Q or C, and
X¹¹=V, M or F,
and
Z¹=0, Z², or pyro glutamate, wherein
Z²=0 or is a modification of the N-terminal nitrogen atom of the peptide chain which modification forms together with the amino group of the N-terminal amino acid of the peptide a moiety having the structure —NR²R³ wherein R² and/or R³ are independently from each other H or a substituted or unsubstituted acyl alkyl, aryl, aralkyl, cyclo alkyl and heterocyclo alkyl group;
Z³=0, or Z⁴, wherein
Z⁴=0 or is a modification of the C-terminal carboxyl group of the peptide chain, which modification forms together with the carboxyl group of the C-terminal amino acid of the peptide a moiety having the structure —C(O)—O—R¹ or —C(O)—NR²R³, wherein R¹ is a substituted or unsubstituted alkyl, aryl, aralkyl, cyclo alkyl and heterocyclo alkyl group.

7. The composition of claim 6, wherein a medicament is selected and comprises a pharmaceutically acceptable carrier, and is suitable for oral, intravenous, intramuscular, intracutaneous, subcutaneous, intrathecal administration or in form of an aerosol suitable for transpulmonary administration, encapsulated in liposomes; or for use in aqueous or liposomal packaging.

8. The composition of claim 6, wherein the polynucleotide is selected from the group consisting of a medicament, a vector, a genetically engineered host cell containing the vector, or a diagnostic agent, comprising at least one polypeptide or polynucleotide coding for the polypeptide.

9. (canceled)

10. The composition of claim 6, wherein the vector containing the polynucleotide is selected from the group consisting of a medicament, a vector, a genetically engineered host cell containing the vector, or a diagnostic agent, comprising at least one polypeptide or polynucleotide coding for the polypeptide.

11. The composition of claim 6, wherein the genetically engineered host cell containing the vector is selected from the group consisting of a medicament, a vector, a genetically engineered host cell containing the vector, or a diagnostic agent, comprising at least one polypeptide or polynucleotide coding for the polypeptide.

12. (canceled)

13. (canceled)

14. (canceled)

15. A method for treatment of a condition, the condition selected from the group consisting of: stroke, Parkinson's disease, Alzheimer's disease, multiple sclerosis, WHIm syndrome, lupus erythematosus, rheumatoid arthritis, cancer, cancers showing the CRCX receptor, cancer of the liver, pancreas, prostate, or breast cancer, lack of mobilization, proliferation and migration of stem cells, T-cell activation, support of immunoblasts, wounds caused by burning, antifibrosis, treatment or prevention of scars, cardiologic disorders, metabolic disorders, diabetes, viral diseases, infections with HIV-I, HIV-2, Cytomegalo virus, Herpes simplex virus (type 1 and 2), Varicella zoster virus, Hepatitis A and Hepatitis B virus, Influenza virus, Polio virus, Rhino virus, Rubella virus, Measles virus, Rabies virus, Rous sarcoma virus, Epstein-Barr Virus, and infections caused by bacteria and fungi, Pseudomonas, Candida, S. aureus, infectious processes, abnormal infectious processes, growth disorders, neuronal diseases, disorders of the blood clotting cascade and hematopoiesis, vascular diseases, diseases of the immune system, or for improving wound and bone healing;
comprising:
administering a medicament comprising a polypeptide and a pharmaceutically acceptable carrier; the polypeptide comprising:
at least one polypeptide having the general amino acid sequence written in the single letter code
Z¹ X¹ X² X³ X⁴ X⁵ Q X⁷ A P X¹⁰ X¹¹ S Z³,
wherein
X¹=V, M or L,
X²=V, L or M,
X³=R, K or C,
X⁴=Y or W,
X⁵=S or T,
X⁷=K, C or R,
X¹⁰=Q or C, and
X¹¹=V, M or F,
and
Z¹=0, Z², or pyro glutamate, wherein
Z²=0 or is a modification of the N-terminal nitrogen atom of the peptide chain which modification forms together with the amino group of the N-terminal amino acid of the peptide a moiety having the structure —NR²R³ wherein R² and/or R³ are independently from each other H or a substituted or unsubstituted acyl alkyl, aryl, aralkyl, cyclo alkyl and heterocyclo alkyl group;
Z³=0, or Z⁴, wherein
Z⁴=0 or is a modification of the C-terminal carboxyl group of the peptide chain, which modification forms together with the carboxyl group of the C-terminal amino acid of the peptide a moiety having the structure —C(O)—O—R¹ or —C(O)—NR²R³, wherein R¹ is a substituted or unsubstituted alkyl, aryl, aralkyl, cyclo alkyl and heterocyclo alkyl group.

16. The polypeptide or polynucleotide coding for the polypeptide according to claim 1, prepared by a process comprising
extracting peptides from hemofiltrate by cation-exchange extraction followed by eluting of the adsorbed substances,
performing renewed cation-exchange chromatography of the extract containing the peptides, and
performing fractional reverse-phase chromatography.

17. The polypeptide or polynucleotide coding for the polypeptide according to claim 1 prepared by a process comprising solid-phase synthesis in terms of Merrifield synthesis or liquid-phase synthesis using protected amino acids, and purifying the product.

18. (canceled)

19. The composition of claim 6, wherein the diagnostic agent is selected, and further comprises poly- or monoclonal antibodies or the nucleic acid or mRNA coding for the polypeptide.

20. The composition of claim 6, wherein the diagnostic agent is selected.

21. The composition of claim 6, wherein the diagnostic agent is selected, and is detectable in tissue, plasma, urine and cerebrospinal fluid levels of this substance by means of mass-spectrometric methods, such as MALDI-MS or ESI-MS.

22. The composition of claim 21, wherein the diagnostic agent is selected and functions as a marker for viral diseases, bacterial and fungal infections, inflammatory and neoplastic processes, inflammatory processes, disturbed inflammation reactions, tumor diseases, growth disorders, diseases of the immune system, or bone diseases.

23. The polypeptide of claim 1, wherein
X¹=L
X²=V
X³=R
X⁴=Y
X⁵=T
X⁷=K
X¹⁰=Q, and
X¹¹=V.

24. The composition of claim 6, wherein a medicament is selected and comprises a pharmaceutically acceptable carrier.

25. The method of claim 15, wherein
X¹=L
X²=V
X³=R
X⁴=Y
X⁵=T
X⁷=K
X¹⁰=Q, and
X¹¹=V.


SEQUENCE LISTING (15 sequences)

SEQ ID NO: 1 - protein, 12 aa, human
LVRYTQKAPQVS

SEQ ID NO: 2 - protein, 12 aa, human
MVRYTQKAPQVS

SEQ ID NO: 3 - protein, 12 aa, human
VVRYTQKAPQVS

SEQ ID NO: 4 - protein, 12 aa, human
LMRYTQKQPQVS

SEQ ID NO: 5 - protein, 12 aa, human
LLRYTQKQPQVS

SEQ ID NO: 6 - protein, 12 aa, human
LVKYTQKQPQVS

SEQ ID NO: 7 - protein, 12 aa, human
LVCYTQKQPQVS

SEQ ID NO: 8 - protein, 12 aa, human
LVRWTQKAPQVS

SEQ ID NO: 9 - protein, 12 aa, human
LVRYSQKQPQVS

SEQ ID NO: 10 - protein, 12 aa, human
LVRYTCKAPQVS

SEQ ID NO: 11 - protein, 12 aa, human
LVRYTQCAPQVS

SEQ ID NO: 12 - protein, 12 aa, human
LVRYTQRAPQVS

SEQ ID NO: 13 - protein, 12 aa, human
LVRYTQKAPCVS

SEQ ID NO: 14 - protein, 12 aa, human
LVRYTQKAPQMS

SEQ ID NO: 15 - protein, 12 aa, human
LVRYTQKAPQFS